(19) United States
(12) Patent Application Publication
(10) Pub. No.: US 2004/0157940 A1
(43) Pub. Date: Aug. 12, 2004

(54) Method of transport energy

(76) Inventor: Robert C. Dalton, Long Beach, NJ (US)

Correspondence Address: TECHNOPROP COLTON, L.L.C., P O BOX 567685, ATLANTA, GA, 311567685

(21) Appl. No.: 10/360,692

(22) Filed: Feb. 7, 2003

(51) Int. Cl. C07C 27/26

(52) U.S. Cl. 518/726

(57) ABSTRACT

A method is used to increase the energy content of a pipeline and other vessels that are designed to carry natural gas under ambient conditions, in a compressed state or in a liquefied state. Methane and other gas are used as the feedstock. Methane from natural gas fields, coal beds or derived from hydrogen reacting with coal are primary energy sources for this method. Also, this method could provide an abundant source for hydrogen production. Energy from hydrogen is used for fuel cell applications that generate electricity and power motor vehicles. This method is capable to increase the energy capacity of current natural gas pipelines and other storage and transport vessels.

### BACKGROUND OF INVENTION

1. Field of Invention

This invention relates generally to a method of transportation of energy and more specifically to a method of transportation of energy that increases the amount of energy in a pipeline or transportation vessel designed to carry methane and other gases with low heats of combustion.

2. Prior Art

Methane, commonly known as natural gas, is a worldwide source of energy. In recent years, natural gas has been a growing source of energy for the United States and other countries. The future consumption of natural gas for energy needs is projected to grow at a fast pace. For example, in 2001, the US government under the Presidency of George W. Bush as well as the US Department of Energy (DoE) stated that the projected use of natural gas as an energy source in the US would grow dramatically as a fuel for the production of electricity. Demand for natural gas for electricity production is expected to rise 90% between 2001 and 2020. However, many bottlenecks exist in natural gas pipelines throughout the US and natural gas pipelines between the US and Canada are at capacity. See Reliable, Affordable, and Environmentally Sound Energy for America's Future, Report of the National Energy Policy Development Group, to President George W. Bush, May 16, 2001 by Dick Cheney, Vice President, pp. 1-5 through 1-8. Furthermore as a new direction to the US energy policy, the US President Bush issued a goal in the President's State of the Union Address on 28 Jan. 2003 to have vehicle transportation fueled by hydrogen. Both the US government's energy policy and the US presidential goals are driven by current and future environmental, economic and national security concerns.

Electricity generation in the US is dominated by coal; however, the future of electricity generation is projected to have a growing demand for natural gas. Today, electricity generation in the US is approximately 52% coal, 20% nuclear, 16% natural gas, 7% hydroelectric, and the balance from oil and renewable energy sources such a wind, solar and biomass. By 2020, the US DoE and the Bush Administration's National Energy Policy Development Group project that the US will become more dependent on energy from natural gas for electricity, transportation, industrial processing, and home heating. These sources predict that electricity from natural gas will increase from today's 16% of generation to 33% of the generation by the year 2020. See Reliable, Affordable, and Environmentally Sound Energy for America's Future, Report of the National Energy Policy Development Group, to President George W. Bush, May 16, 2001 submitted by Dick Cheney, Vice President.

For electricity production, methane is considered an abundant source of energy, is environmentally advantageous over coal, is more energy efficient for electricity production with lower-capital equipment costs and shorter construction lead times for electricity plants, and is favored by power generation companies due to changes in the economics of electricity generation. Methane is an abundant natural resource for energy within the US and around the world. Several experts estimate the amount of natural gas that is located off the eastern seaboard of the US to be over 100 trillion cubic feet (tcf). Other abundant sources are gas methane associated with coal beds and methane produced from coal. The US has coal reserves that would last over 200 years, and a large amount of methane gas that is found in coal beds is being captured today. Other estimates of natural gas reserves around the world are large. Estimates of quantities of known stranded gas reserves have been identified by synthetic fuel manufactures such as Syntroleum, Inc. Syntroleum, Inc. places the quantity of these stranded gas reserves to be equivalent to the oil reserves of Saudi Arabia, if these reserves were converted from methane gas to hydrocarbon liquids fuels. These reserves would provide hydrocarbon liquid fuel from Fischer-Tropsch synthesis methods that could provide fuel for all the cars and trucks in the US for over 80 years. Other methods for methane production include reacting hydrogen with coal.

Besides its abundance for an energy source, methane is sought after for environmental, economical and energy efficiency reasons. Shifting energy sources for electricity generation to natural gas (methane) provides many environmental advantages compared to coal and nuclear sources. When methane is used as a primary source for electricity and energy produced by gas turbines, electricity generation produces fewer emissions that lead to pollution and poor air quality, compared to coal. Unlike coal, electricity generation from methane-fired gas turbines produces low emissions of nitrogen oxides (NOₓ) and sulfur dioxides (SO₂) and virtually no emissions of organic particulates, chloride, fluorides, mercury, hazardous metals, and other pollutants. In addition, electricity production from methane produces less carbon dioxide (CO₂) emissions than coal. Carbon dioxide emissions are considered among many in the scientific community to cause global warming. Generation of electricity from nuclear energy produces no pollutant or carbon dioxide emissions, but the byproducts from fuel preparation and spent fuel creates environmental hazards. The nuclear fuel manufacturing process introduces a large number of environmentally hazardous chemical and isotopes into the environment, and spent fuel contains highly radioactive byproducts that can last thousands of years.

Another environmental advantage of electricity production from methane compared to coal is that electricity is produced more energy efficiently from natural gas turbines. Electricity generation from natural gas can be very energy efficient. Natural gas-fired turbines can produce electricity with and without cogeneration. Cogeneration can produce either steam or steam and electricity from steam turbines. Cogeneration, also known as combined heat and power (CHP), can achieve efficiency of greater than 80%, whereas the newest coal-burning power plant can achieve efficiencies of only slightly over 40%. However, most conventional coal-fired power plants operate at approximately 30% efficiency.

The future demand for methane's energy is not just being driven by electricity demand. Energy consumption from methane accounts for 24% of the total energy used in the US. Methane is a feedstock for many products and a source of energy for many manufacturing processes. These products include textiles, chemicals, rubber, and furniture. Manufacturing processes that rely heavily on natural gas include brick making, glass making, and paper production. Residential heating produces a great demand for energy from natural gas, also.

According to the Report of the US National Energy Policy Development Group, the Bush Administration cites that:

Between 2000 and 2020, U.S. natural gas demand is projected by the Energy Information Administration to increase by more than 50 percent, from 22.8 to 34.7 trillion cubic feet. Others such as the Cambridge Energy Research Associates expect gas consumption to increase by about 37 percent over that period. Growth is projected in all sectors—industrial, commercial, residential, transportation, and electric generation. More than half of the increase in overall gas consumption will result from a rising demand for electricity generation.

The report further cites current and future problems associated with getting methane's energy to the market place:

To meet this long-term challenge, the United States not only needs to boost production, but also must ensure that the natural gas pipeline network is expanded to the extent necessary. For example, although natural gas electricity generation in New England is projected to increase by 16,000 MW through [2020], bottlenecks may block the transmission of necessary supplies. Unless pipeline constraints are eliminated, they will contribute to supply shortages and high prices, and will impede growth in electricity generation.

The report further cites that:

The current domestic natural gas transmission capacity of approximately 23 trillion cubic feet (tcf) will be insufficient to meet the projected 50 percent increase in U.S. consumption projected for 2020. Some parts of the country such as California and New England, already face capacity shortage. . . . [D]elays have constrained the ability to transport natural gas to California, contributing to high prices. In addition, the natural gas pipeline connections from Canada are near capacity, so any greater U.S. reliance on Canadian natural gas will require increase pipeline capacity.

Transportation of methane's energy is cited as one of the major hurdles for meeting the projected demands for natural gas. The expected increase in the demand for methane's energy is expected to require 263,000 miles of distribution pipelines and 38,000 miles of new transmission pipelines. Construction of these miles of pipelines will face obstacles. These obstacles are, but are not limited to, encroachment on existing rights-of-ways and heighten community resistance to pipeline construction.

Liquefying methane is one method to increase methane's energy density for transportation of the energy of methane. By liquefying natural gas, the energy that is contained in one thousand cubic meters (1000 m³) of methane gas at standard temperature and pressure is compressed into approximately a volume of one cubic meter (1 m³) in the liquid state of methane. Liquefied natural gas (LNG) can be transported through pipelines or transported by specially designed ships. Ships commonly transport liquefied natural gas. Transportation by ship uses liquefied natural gas to increase the energy density of the ship's storage volume increasing the amount of energy that the ship can carry. This above stated increase demand for methane's energy to generate electricity could require a substantial demand for LNG imports. The current demand for methane's energy has begun to demonstrate this trend. The current market in New England has seen a 350% increase in imports LNG from ship between 1998 and 1999. Several companies have considered reopening terminals in Georgia and Maryland to import LNG. Other petroleum companies have announced plans for creating terminals to import LNG.

Constraints from transportation of methane's energy are not limited to pipelines; ships are also subject to constraints. Common factors can inhibit shipping of LNG to certain regions of the US. In the Mid-Atlantic and New England States shipping of LNG can be difficult in the winter months. Cold weather can ice over harbors and rivers. Frozen marine channels, rivers and harbors affect marine transport of LNG that become problematic when demand is great for heating at cold temperatures.

Conventional facilities to liquefy methane tend to be quite large and expensive to build. Hundreds of millions of US dollars are typically required to build a LNG process facility. Newer technology has decreased the cost of LNG processing facilities. One such new technology is small, natural gas driven compressors invented by the US DoE at Los Alamos National Laboratory (LANL). The technology is called thermoacoustic natural gas liquefaction. Among patents for this technology are U.S. Pat. No. 4,953,366 by Swift et al. and U.S. Pat. No. 4,858,441 by Wheatley et al. This technology is also known as Thermoacoustic Sterling Hybrid Engine Refrigerator (TASHER).

The US DoE and its industrial partner have spent over US$20 million to demonstrate this thermoacoustic technology. The technology is quite small and effective for liquefying natural gas. The main markets for this technology are liquefying methane on drilling platforms at sea for transporting by ship, liquefying stranded coal-bed methane for transportation by pipeline, rail car or truck, and liquefying natural gas at the end-of-pipe/end-of-line or at-the-market locations to increase the energy content of fuel containers that are used for vehicle transportation that operate on methane's energy.

Another prior art method to transport methane's energy is to convert methane gas to liquid fuel using steam reforming with Fischer-Tropsch catalysts and autocatalytic oxidation of methane. This method is quite common to transport stranded methane gas and is sought after to increase the pressure on oil pipelines to transport oil from mature oil fields where oil production is declining. Stranded methane gas is methane gas that has no common economic means to be transported from remote locations to the market place. For example, locations where no pipelines exist to transport the natural gas to ports or the market place.

Gas-to-hydrocarbon liquid technologies and processes have received much attention by the US DoE to supplement the constant decline in oil from Alaska's North Slope with Fischer-Tropsch methods. The hydrocarbon liquid fuels derived from methane are intended to keep the pressure on the Alaskan Pipeline great enough to transport the remaining oil in the North Slope as production continues to decline. Other companies, such as Syntroleum, Inc., use autocatalytic oxidation of methane to produce liquid fuel with ultra-low sulfur contents as additive to common gasoline to meet new US Environmental Protection Agency (EPA) sulfur standards for gasoline and conventional diesel fuels. Syntroleum, Inc. has received many US patents in this area, including U.S. Pat. No. 6,344,491 for a high-pressure autothermal oxidative catalytic process for methane and U.S. Pat. No. 6,085,512 for other Fischer-Tropsch technology.

Other methods and technologies to transform and transport methane's energy by converting methane gas into a liquid hydrocarbon fuel by the US DoE and their industrial and university partners include Ion Transport Ceramic Membrane and Steady-State & Transient Catalytic Oxidation and Coupling of Methane. See, e.g., www.fe.doc.gov/fuel/gas-to-liquids.shtml.

Other methods to increase the amount of methane's energy available to the market place use prior art that is associated with current energy policy and conventional energy transportation methods. These methods provide a reasonable, conventional solution to addressing the constraints of delivering methane's energy to the market place. One method is to build more pipelines—distribution pipelines and transmission pipelines. One other is to increase the energy content of a methane gas pipeline is to increase the pressure of the gas in the pipeline. These conventional approaches would, as stated in the Report of the US National Energy Policy Development Group, call for increasing the amount of energy supplied from methane by building tens of thousands of miles of new transmission pipelines and hundreds of thousands of miles of new distribution pipelines. The cost for the new infrastructure to transport the energy of methane is estimated to be well over US$10 billion.

Other prior art contains end-of-the-pipe/end-of-the-line or at-the-market technology to process natural gas, methane. These technologies convert methane to chemical species for feedstock to other process for an end use. Such uses include feedstocks such as ethane and ethylene for plastics such as polyethylene and polypropylene. Other technologies are used to convert methane to acetylene as well as to use methane for gas-to-hydrocarbon liquid processes. These technologies employ processes that use catalysts, electromagnetic energy, non-thermal plasma and plasma initiators. Some technologies use these processing in combination with each other. These technologies use methane, coal, carbon sources, water and hydrogen as input chemicals species for producing feedstock chemical for industrial process. Methane can be processed with coal, a carbon species, or a carbon containing species. Methane also can be processed alone, with water, or with hydrogen or oxygen. Coal can be processed with hydrogen, water, or hydrogen with water.

Other prior art processes include U.S. Pat. Nos. 5,328,577 and 5,277,773 to Murphy disclose the use of plasma initiators exited by microwave energy to convert methane and hydrogen to acetylene, ethylene, and ethane. U.S. Pat. No. 5,972,175 to Tanner discloses the use of a catalyst heated with microwave energy to convert gaseous hydrocarbons, methane and propane, with char to synthesize higher order organic species including ethylene and acetylene.

U.S. Pat. No. 4,574,038 to Wan discloses processing 100% methane with microwave energy and a metal catalyst to produce a product mixture of 51.3% ethylene, 21.8 methane and 26.7 hydrogen. U.S. Pat. No. 5,472,581 to Wan discloses the use of microwave energy to heat activated charcoal to react the charcoal with methane to produce ethane, ethylene and acetylene. Also, Wan '581 discloses the use of microwave energy to heat activated charcoal with water to produce methane, ethane, ethylene and acetylene.

U.S. Pat. No. 5,900,521 to Park discloses creating a metal catalyst that uses a conventionally heated catalysts bed to convert methane to ethylene and hydrogen. U.S. Pat. Nos. 5,131,993 and 5,015,349 to Suib disclose the use of a non-thermal plasma, catalyst and microwave energy to synthesize higher order hydrocarbons from methane.

Bool et al. have used microwave energy as a catalyst to react oxygen and methane to form ethylene, carbon monoxide and acetylene. Bool, C. J. et al, The Application of Microwaves to the Oxidative Coupling of Methane over Rare-Earth Oxide Catalyst, source unknown, pp. 39-42, School of Chemistry, University of Hull, Hull, North Humberside, United Kingdom, HU67RX.

These many processes produce higher energy gases from methane, methane and coal, methane and water, methane and oxygen, methane and hydrogen, coal and hydrogen, and coal and water that have higher heats of combustion compared to methane and that have higher boiling points compared to methane. Compared to methane alone, these mixtures of gases have a lower number of moles if the hydrogen is remove from the mixture.

Even with these methods, there is a need for a more efficient method of transporting methane and other gases to as to provide a higher energy content in a smaller volume of gas. It is to this need and other needs that the present invention is directed.

### BRIEF SUMMARY OF THE INVENTION

One aspect of this invention is to increase the amount of energy that can be transported through (1) a pipeline or (2) in a storage vessel by synthesizing high energy gases (HEG) from a lower energy gas, then transporting these higher energy gases through conventional energy transportation methods such as for example, but not limited to, gas pipelines, liquefied gas pipelines, high pressure vessel, etc. The invention involves the conversion of low energy gases such as methane and syngas (CO and H₂) to higher order molecules. These higher order gases have greater heats of combustion compared to methane and other gases and gas mixtures. Also, these high-energy gases have higher boiling points that would require less energy to condense them into a liquid.

The typical byproduct of synthesizing high-energy gases is hydrogen (H₂). Hydrogen is consider an environmentally friendly source of energy, is a future source of energy for electricity from fuel cells and for a clean burning fuel source for motor vehicles, and supports the efforts of the US to build a hydrogen economy for energy. This invention addresses the current and projected shortfalls of infrastructure to transport an abundance of methane's energy for the energy and environmental needs of the US, and the world, and addresses the future demands for a clean burning source of energy, such as hydrogen.

### BRIEF DESCRIPTION OF THE DRAWINGS

FIG. 1 is a flow diagram for the basic method for high energy gas (HEG) transport.

FIG. 2 is a Flow Diagram for High Energy Gas (HEG) Transport with Hydrogen Separation.

FIG. 3 is a Flow Diagram for High Energy Gas (HEG) Transport with Methane Recycling and Hydrogen Separation

FIG. 4 is a Flow Diagram for High Energy Gas (HEG) Transport with Methane and Hydrogen Recycling.

FIG. 5 is a Flow Diagram for High Energy Gas (HEG) Transport with Hydrogen Separation and “Down-the-Pipe” Separation of Methane and High Energy Gases.

FIG. 6 is a Flow Diagram Depicting a Bottleneck in a Distribution Line for Methane.

FIG. 7 is a Flow Diagram Depicting the Application of High Energy Gas (HEG) Synthesis to Remove a Bottleneck in a Distribution Line for Methane.

FIG. 8 is a Flow Diagram Depicting a Bottleneck in a Transmission Line for Methane.

FIG. 9 is a Flow Diagram Depicting the Application of High Energy Gas (HEG) Synthesis to Remove a Bottleneck in a Transmission Line for Methane.

### DETAILED DESCRIPTION OF THE PREFERRED EMBODIMENTS

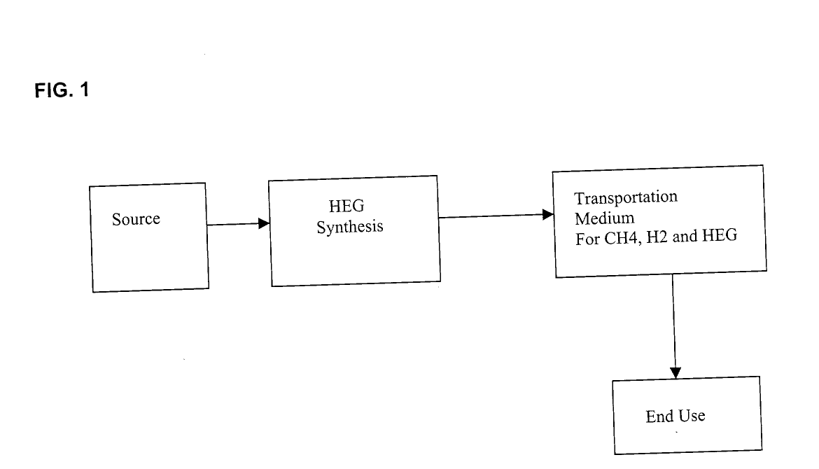

The invention relates to a novel method to transport energy by forming high-energy gases (HEG) then transporting the HEG through conventional transportation means. This transportation method allows for a greater amount of energy to be transported to an end use. As shown in the flow chart of FIG. 1, a source for conventional gases commonly used for providing energy is first treated by a means to synthesize high-energy gases (HEG) and then is transported through conventional and exiting transportation means to the end use of the energy. The source can be for illustrative purposes naturally occurring methane (CH₄), syngas (CO and H₂), a solid carbon source that is reacted with a gaseous or liquid species, methane produce by biomass decomposition, and methane produce from landfill decomposition. When the source is a solid carbon species, the carbon can be reacted with water (H₂O), methane (CH₄), carbon monoxide (CO), carbon dioxide (CO₂), or hydrogen (H₂). The carbon source for illustrative purposes can be coal, char, or biomass. High energy gas (HEG) is a gas that is reformed from a carbon containing species with a heat of combustion that is less than or equal the heat of combustion of methane (890.9 KJ/mole), and the synthesized high energy gas has a heat of combustion that is greater than methane (CH₄). The high energy gas (HEG) can be one gas or a mixture of gas.

Table 1 lists the heat of combustion for gases that could be used to synthesize high-energy gases (HEG) and gases that are high-energy gases (HEG). Carbon monoxide (CO), hydrogen (H₂), and methane (CH₄) are source gases used to form high-energy gases (HEG). Acetylene (C₂H₂), ethylene (C₂H₄), ethane (C₂H₆), and propylene (C₃H₆) are high energy gases. It is possible that other gases can be synthesized with heats of combustions that are greater than methane. These other gases are also high energy gases, and it is understood that the above stated high energy gases can be recycled into the means to synthesize HEG to reform the recycled gas into a HEG with heats of combustion greater than acetylene.

TABLE 1

| | Heat of Combustion |
|---|---|
| Gas | (Δ_{c}H/KJmol-1) |
| Carbon Monoxide, CO | 283.0 |
| Hydrogen, H₂ | 285.8 |
| Methane, CH₄ | 890.8 |
| Acetylene, C₂H₂ | 1301.1 |
| Ethylene, C₂H₄ | 1411.2 |
| Ethane, C₂H₆ | 1560.7 |
| Propylene, C₃H₆ | 2058.0 |

High-energy gases from source gases or source gases that are reacted with a solid carbon species can be synthesized by known means such as, for example purposes only and not limited to those disclosed in U.S. Pat. No. 4,574,038 to Wan, U.S. Pat. No. 5,972,175 to Tanner, U.S. Pat. No. 5,900,521 to Park, and U.S. Pat. Nos. 5,131,993 and 5,015,349 to Suib, all of which are incorporated herein by reference. These methods include means that use autothermal catalysis, thermal catalysis, electromagnetic energy, plasma, steam reforming, and others. After the HEG is synthesized the HEG is transported to the end use or user as shown in FIG. 1. The transportation means are conventional transportation means and methods, including but not limited to transmission pipelines, distribution pipelines, high-pressure vessels, liquefaction, and other transportation and storage methods.

In this invention, HEG can be transported in a mixture with methane and hydrogen by conventional transportation means. This invention allows for a greater amount of energy to be transported by convention transportation means.

Table 2 provides examples of mixtures of high-energy gases (HEG) and the associated amount of energy with 10 moles of each mixture. Table 2 also provides a normalized energy content for the 10 moles of gases. The normalized energy content is normalized to the amount of energy from the conventional method of transporting gaseous energy in natural gas, methane. The total heat of combustion (Δ_{c}H^{o}_{T}) for ten moles (10 mol.) of methane (CH₄) is 8909 KJ.

TABLE 2

| | Total | Normalized | Moles of |
|---|---|---|---|
| Gas Mixture | Heat of | Energy Content | Hydrogen |
| Containing 10 Moles | Combustion | (Δ_{c}H°_{T-Mixture}/ | (H₂) |
| (Mol. of Each Gas) | (Δ_{c}H°_{T}) | Δ_{c}H°_{T-Methane}) | Produced |
| 10 Mol. of Methane, CH₄ | 8909 KJ | 1.00 | 0 |
| 7 Mol. of Methane, CH₄ | | | |
| 3 Mol. of Ethylene, C₂H₄ | 10500 KJ | 1.18 | 6 |
| 5 Mol. of Methane, CH₄ | | | |
| 5 Mol. of Ethylene, C₂H₄ | 11512 KJ | 1.29 | 10 |
| 3 Mol. of Methane, CH₄ | | | |
| 7 Mol. of Ethylene, C₂H₄ | 12551 KJ | 1.41 | 14 |
| 10 Mol. of Ethylene, C₂H₄ | 14112 KJ | 1.58 | 20 |
| 3 Mol. of Methane, CH₄ | | | |
| 3 Mol. of Acetylene, C₂H₂ | 12221 KJ | 1.37 | 17 |
| 4 Mol. of Ethylene, C₂H₄ | | | |
| 2 Mol. of Methane, CH₄ | | | |
| 2 Mol. of Acetylene, C₂H₂ | 14145 KJ | 1.59 | 10 |
| 4 Mol. of Ethylene, C₂H₄ | | | |
| 2 Mol. of Propylene, C₃H₆ | | | |

As shown in Table 2, this invention, which utilizes high-energy gas (HEG) mixtures for transporting energy, allows for a greater amount of energy to be transported by conventional means. When a high energy gas is mixed with methane, the resultant amount of energy is greater than 8909 KJ. The examples range from a mixture of 7 moles of methane with 3 moles of ethylene to 10 moles of ethylene to a mixture of 2 moles of methane with 2 moles of acetylene, 4 moles of ethylene and 2 moles of propylene. These mixtures have total heats of combustion for ten moles of gas that are greater than 10 moles of methane. The heats of combustion for these mixture range from 10500 KJ to 14125 KJ.

When the energy content of these HEG mixtures are normalized against the amount of energy for methane alone, the amount of energy ranges from 18% to 59% greater than methane alone. The greater amount of energy associated with HEG mixtures allows for more energy to be transported through existing conventional pipelines. As stated by the US Report of the National of the National Energy Policy Development Group, between the years 2000 and 2020 the demand for energy from natural gas is expected to rise between 37% and 50%. And according to this report, this increase in energy demand is expected to require over 38,000 miles of new transmission pipelines and 263,000 miles of new distribution pipelines, and this new pipeline construction is expect to cost well over US$10 billion.

As shown in Table 2, the use of HEG allows for more energy to be transported compared to conventional means, thus energy associated with mixtures of HEG can eliminate or substantially reduce the need for new pipelines resulting in saving billions of US dollars in pipeline construction. With HEG mixtures, more energy is transported more efficiently to the end users to meet growing energy demands without significantly increase the transportation infrastructure. This invention is similar to transmitting more data over optic fiber lines by increasing the capacity by using more that one frequency to transport data. Whereas multimode optics increase the capacity of single optic fiber, HEG increases the energy capacity of a pipeline or other convention transportation means (i.e., a ship that carries liquefied natural gas, LNG). This invention also has the potential to reduce the cost of electricity and home heating by reducing transmission costs by increasing the energy capacity of a pipeline.

An ancillary benefit of this invention is the abundant production of hydrogen for an energy economy based upon hydrogen. Hydrogen is expected to be in demand as an environmentally friendly energy fuel source for producing electricity from fuel cells and to power motor vehicles. As exemplified in Table 2, HEG mixtures with and without methane produce hydrogen. In these examples the HEG mixtures produce between 6 moles and 20 moles of hydrogen (H₂) based upon synthesizing 10 moles of the HEG mixture. The amount of hydrogen was obtained by converting methane to the HEG mixture. An example of a calculation for hydrogen produced is given below:

20CH₄→2CH₄+2C₂H₂+4C₂H₄+2C₃H₆+10H₂ Equation (1)

Or

20 mol. Methane→2 mol. methane+2 mol. Acetylene+4 mol. ethylene+2 mol. propylene+10 mol. hydrogen Equation (2)

The reaction above can be produced by the mentioned HEG synthesis methods with a recycling of non-reformed methane through a reactor.

Another benefit from this invention is energy savings on liquefying gas. As shown in Table 3, the boiling point of the HEG is greater than methane. Acetylene, ethylene, ethane and propylene all have higher boiling points compared to methane. These higher boiling points would allow for a high-energy gas or a mixture of high-energy gases without methane or hydrogen to be compressed into a liquid with less energy.

TABLE 3

| Gas | Boiling Point (° K) |
|---|---|
| Carbon Monoxide, CO | 81.6 |
| Hydrogen, H₂ | 20.28 |
| Methane, CH₄ | 111.6 |
| Acetylene, C₂H₂ | 188.4 |
| Ethylene, C₂H₄ | 169.4 |
| Ethane, C₂H₆ | 184.5 |
| Propylene, C₃H₆ | 225.5 |

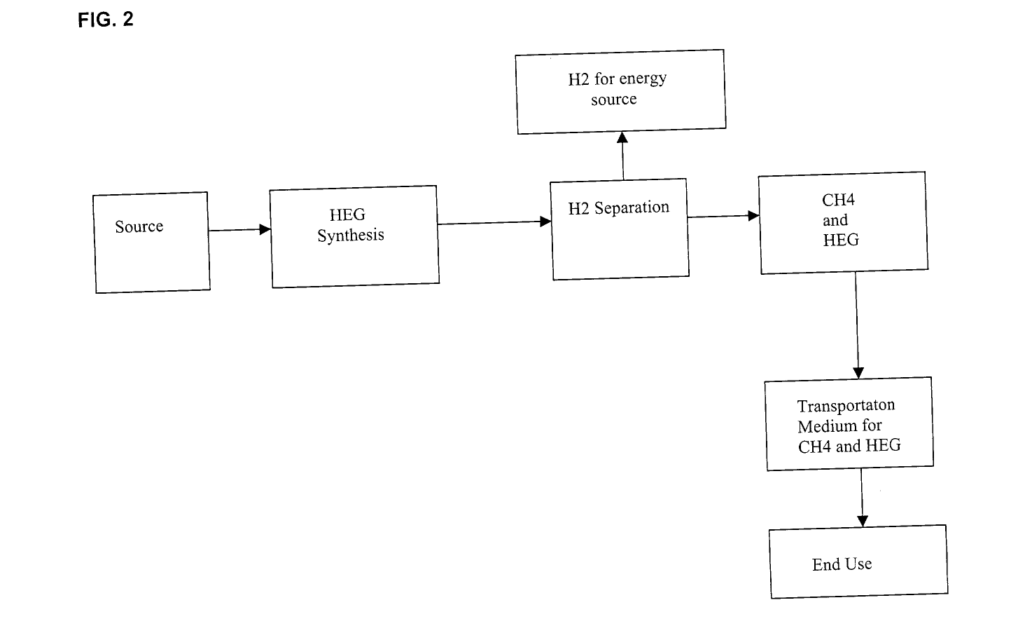

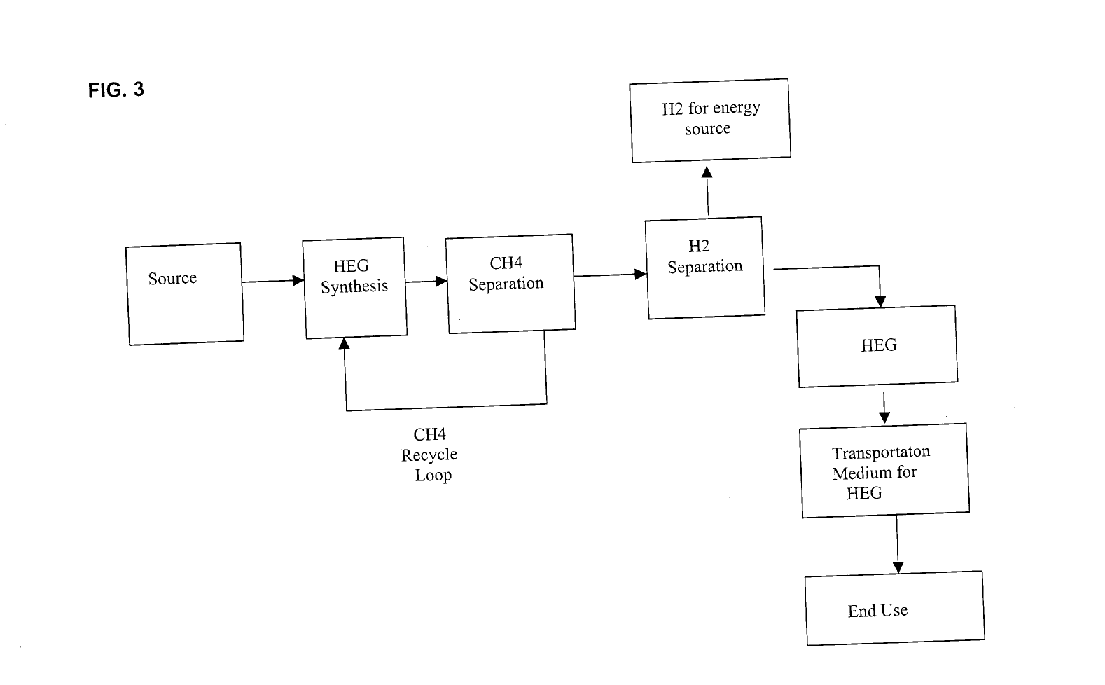

This invention can use variations to transport energy. The flow chart of FIG. 2 shows a variation that separate out hydrogen after the HEG synthesis and transports mixture of methane and high-energy gas. The separated hydrogen can be used as an energy source to generate electricity or for fuel for motor vehicles. The flow chart of FIG. 3 shows a variation where some or all the methane can be separated from the products of the HEG synthesis and recycled back into the HEG synthesis process to create high-energy gases. After separating out the methane, hydrogen is removed from the HEG prior to transportation.

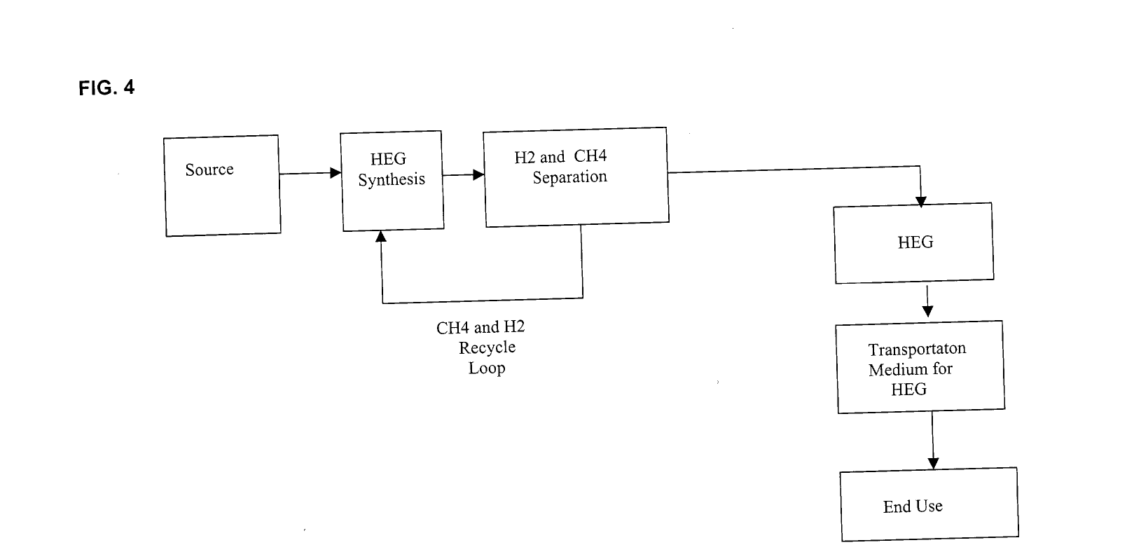

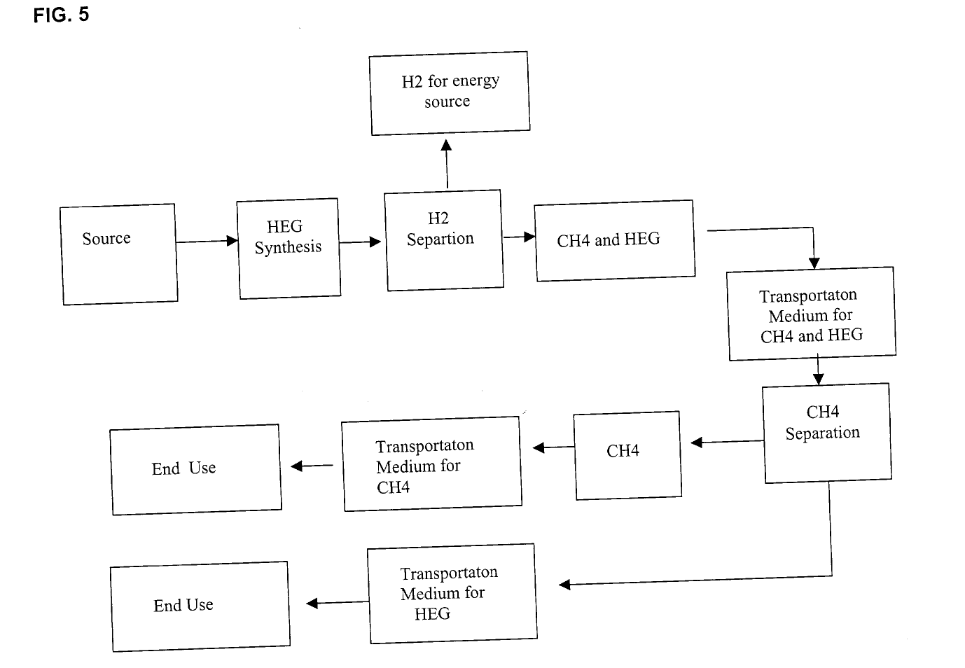

The flow chart of FIG. 4 illustrates a process where some or all of the hydrogen and some or all of the methane are separated from the product of the HEG synthesis method and recycled back into the HEG synthesis process. The HEG or HEG mixture is then transported to the end use. The flow chart of FIG. 5 shows a process where hydrogen is separated from the product stream after the HEG synthesis process. Methane and HEG are transported together. At a further time in the transmission of the mixture, some or all of the methane can be separated out of the transmission method for an intended end use. For example, methane (CH₄) can be separated from the mixture for home heating while the remaining mixture of methane and HEG is used for electricity generation. While not shown, in FIGS. 2 through 5, it should be understood that in the scope of the invention the high energy gases could be mixed with methane during the transmission.

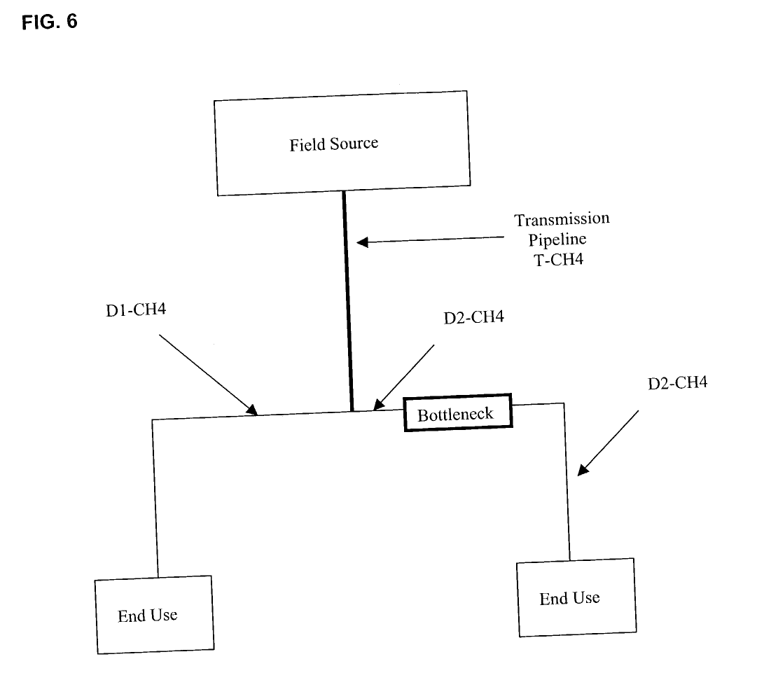

Another benefit of this invention is that bottlenecks in transmission pipelines and distribution pipelines can be eliminated. This invention allows for satellite operations for HEG synthesis to relieve bottlenecks in transportation of energy. The invention would allow move energy to be transmitted to the end uses without having to construct a new pipeline. As shown in the flow chart of FIG. 6, a transmission pipeline T-CH4 carries methane to two (2) distribution pipelines D1-CH4 and D2-CH4, both carrying methane. A bottleneck is present in D2-CH4 that prevents the end use from receiving the amount of energy that is required at the end use.

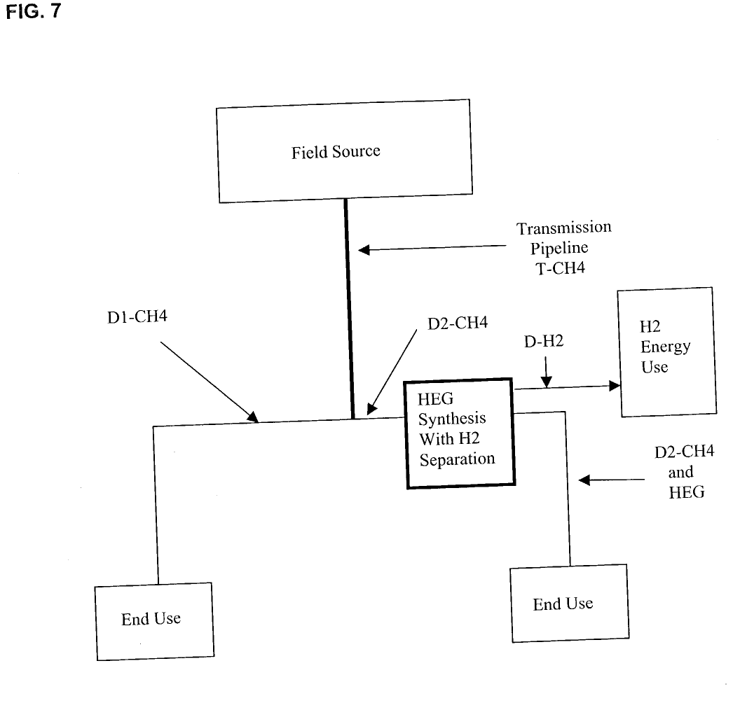

As shown in the flow chart of FIG. 7, the HEG method is used to alleviate the bottleneck in D2-CH4 by providing more energy. Through the invention, the bottleneck is eliminated. After the HEG synthesis process, distribution pipeline line D2-CH4 now carries a mixture of methane and HEG. The distribution pipeline after the HEG synthesis is label D2-CH4 and HEG because it carries more energy from this energy transportation method. Construction of additional distribution pipelines was not required to meet the energy demands of the end use. Also shown in FIG. 7, hydrogen is separated after the HEG synthesis process. As an example, the hydrogen could be used as an energy source for motor vehicles or to generate electricity from a fuel cell.

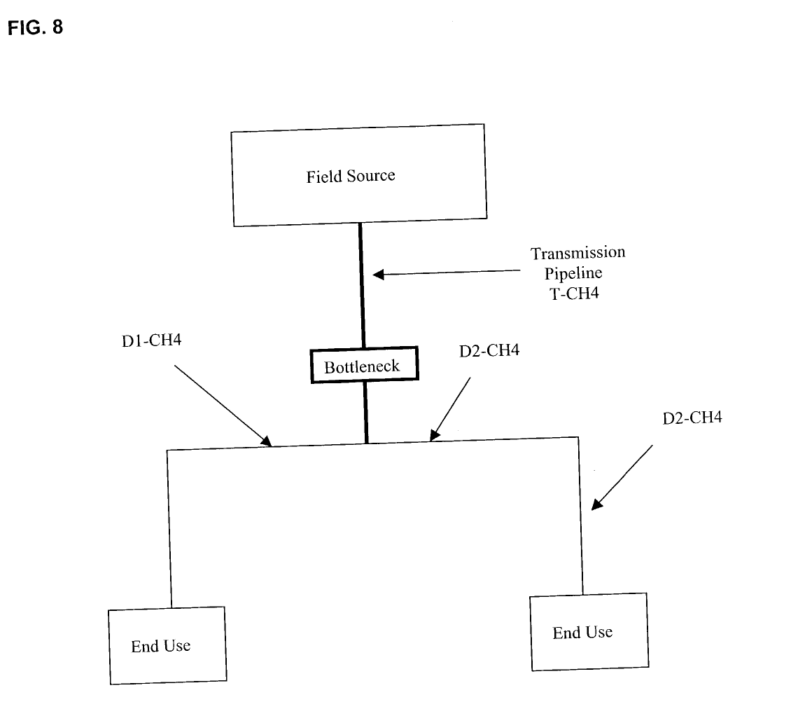

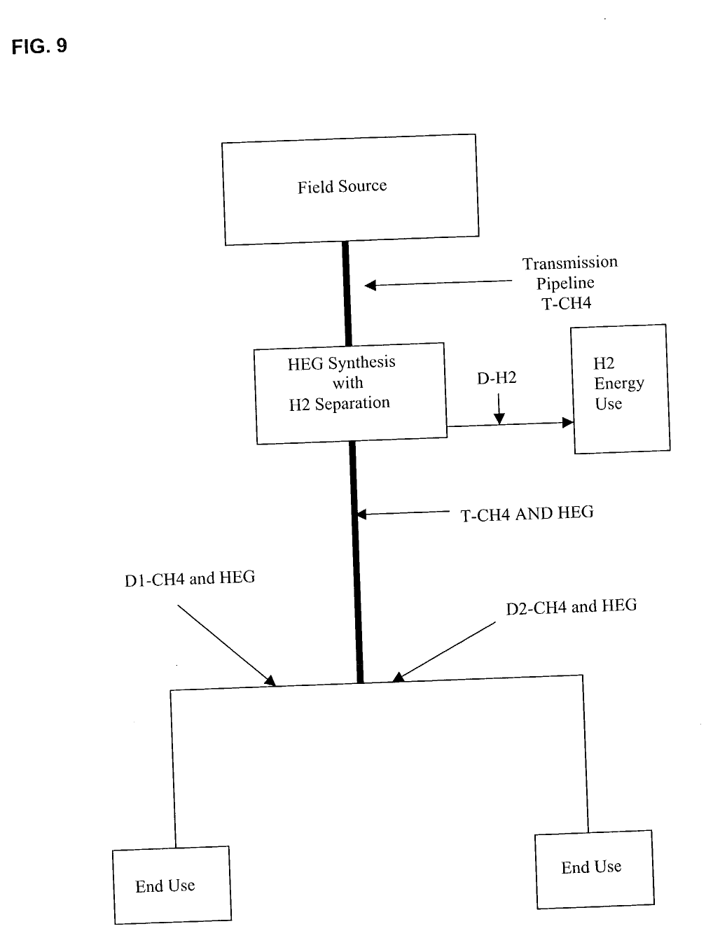

As shown in the flow chart of FIG. 8, a bottleneck is present in transmission pipeline T-CH4. T-CH4 carries methane only. As shown in the flow chart of FIG. 9, the bottleneck is eliminated by using the HEG method to transport a higher-energy capacity through the transmission pipeline. Prior to HEG synthesis transmission pipeline carried methane only T-CH4. After HEG synthesis, the transmission pipeline now carries more energy to meet the demands of the end use. The transmission pipeline is labeled T-CH4 and HEG because it now carries a mixture of high-energy gases and methane. Likewise, the two (2) distribution pipelines are now relabeled, because they both carry a mixture of methane and high-energy gases. One is relabeled D1-CH4 and HEG. The other one is relabeled D2-CH4 and HEG. FIG. 9 also depicts a hydrogen separation process. The hydrogen can be use as an energy source for electricity generation or as a fuel for motor vehicles.

New regulations (deregulation) for electricity transmission and sale of electricity allow for this invention to use abundant and unused excess energy that is available in the US at night from nuclear-powered and coal-powered electricity generation. Unlike energy from electricity, energy from gases can be readily stored at great quantities for later use when demand is great. For example, high-energy gases can be synthesized at night with excess electricity available at night and stored for a later use.

The above description sets forth the best mode of the invention as known to the inventor at this time, and is for illustrative purposes only, as one skilled in the art will be able to make modifications to these methods without departing from the spirit and scope of the invention and its equivalents as set forth in the appended claims.

What is claimed is:

1. A method to transport energy comprising the steps of:
a. providing an input chemical species comprising at least one gas species containing carbon and having a heat of combustion equal to or less than methane;
b. converting at least a portion of the input chemical species flow using a reforming process into an output chemical species that has at least one new gaseous species having a higher heat of combustion than methane, where total number of moles of the input chemical species is greater than the total number of moles of the output chemical species minus the number moles of hydrogen in the output chemical species; and
c. transporting the output chemical species by conventional transportation means to an end use.

2. The method to transport energy as claimed in claim 1, wherein the input chemical species further comprises a component selected from the group consisting of methane, hydrogen, carbon monoxide, carbon dioxide, water, a species containing carbon, and combinations thereof.

3. The method to transport energy as claimed in claim 1, wherein the species containing carbon is a solid.

4. The method to transport energy as claimed in claim 3, where the species containing carbon is selected from the group consisting of coal, activated carbon, char, biomass, and combinations thereof.

5. The method to transport energy as claimed in claim 1, wherein hydrogen is separated from the output chemical species prior to being transported by conventional means to an end use.

6. The method to transport energy as claimed in claim 1, wherein methane is separated from the output chemical species and recycled back into the input chemical species for reforming.

7. The method to transport energy as claimed in claim 1, wherein hydrogen is separated from the output chemical species and recycled back into the input chemical species for reforming.

8. The method to transport energy as claimed in claim 1, wherein hydrogen and methane are separated from the output chemical species and recycled back into the input chemical species for reforming.

9. The method to transport energy as claimed in claim 1, wherein the process is optimized to produce hydrogen.

10. The method to transport energy as claimed in claim 1, wherein the process is optimized to maximize total heat of combustion of the output chemical species.

11. The method to transport energy as claimed in claim 1, wherein the transported energy contains at least a portion of methane.

12. The method to transport energy as claimed in claim 1, wherein the transported energy does not contain methane.

13. The method to transport energy as claimed in claim 1, wherein the method is employed to reduce a bottleneck in a transmission pipeline or a distribution pipeline.

14. The method to transport energy as claimed in claim 1, wherein hydrogen is separated from the output chemical species and the hydrogen is used as an energy source to generate electricity or to fuel a motor vehicle.

15. The method to transport energy as claimed in claim 1, wherein the output chemical species has a higher heat of combustion than methane and comprises a compound selected from the group consisting of acetylene, ethylene, ethane, propylene, and combinations thereof.

16. The method to transport energy as claimed in claim 1, wherein the output chemical species has a heat of combustion greater than acetylene.

17. The method to transport energy as claimed in claim 1, wherein a portion of the output chemical species having a heat of combustion equal to or greater than acetylene is separated from the output chemical species and is recycled back into the input chemical species.

18. A method to transport energy comprising the steps of:
a. providing an input gas species comprising (i) a component selected from the group consisting of methane, hydrogen, carbon monoxide, carbon dioxide, water, a species containing carbon, and combinations thereof, and (ii) at least one gas species containing carbon and having a heat of combustion equal to or less than methane;
b. converting at least a portion of the input chemical species flow using a reforming process into an output chemical species that has at least one new gaseous species having a higher heat of combustion than methane, where total number of moles of the input chemical species is greater than the total number of moles of the output chemical species minus the number moles of hydrogen in the output chemical species; and
c. transporting the output chemical species by conventional transportation means to an end use.

19. The method to transport energy as claimed in claim 18, wherein the species containing carbon is a solid selected from the group consisting of coal, activated carbon, char, biomass, and combinations thereof.

20. The method to transport energy as claimed in claim 18, wherein a portion of the output chemical species having a heat of combustion equal to or greater than acetylene is separated from the output chemical species and is recycled back into the input chemical species.
